(19) United States
(12) Patent Application Publication
(10) Pub. No.: US 2024/0257917 A1
(43) Pub. Date: Aug. 1, 2024

(54) SYSTEM AND METHOD FOR REDUCING A NUMBER OF TESTINGS FOR A HIGH DIMENSIONAL ASSAY

(71) Applicant: ALGORITHMIC BIOLOGICS PRIVATE LIMITED, Bangalore (IN)

(72) Inventor: Manoj Gopalkrishnan, Mumbai (IN)

(21) Appl. No.: 18/014,335

(22) PCT Filed: Dec. 17, 2022
(86) PCT No.: PCT/IN2022/051091

§ 371 Date: Jan. 3, 2023

(30) Foreign Application Priority Data
Dec. 17, 2021 (IN) 202141059066

(51) Int. Cl. G16B 40/10

(52) U.S. Cl. CPC G16B 40/10

(57) ABSTRACT

The present invention provides a system (200) for reducing a number of testings for a high-dimensional assay for detecting, identifying, and quantifying a plurality of analytes in a plurality of biological samples. The system is configured to (i) generate a pooling matrix for pooling and testing the plurality of biological samples, (ii) obtain an output data on completing the high-dimensional assay in each of the plurality of pools, (iii) generate a set of linear equations based on the output data and the generated pooling matrix, and (iv) convert the set of linear equations using a compressed sensing algorithm and at least one regularity condition to detect, identify, and quantify the plurality of analytes in the plurality of biological samples. The regulatory condition is sparsity with respect to a presence or an absence of each analyte separately, or a disproportionate number of samples having disproportionately high values for a particular analyte.

### CROSS-REFERENCE TO RELATED APPLICATIONS

This patent application is related to pending Indian patent application no. 202141044465 filed on Sep. 30, 2022, and Indian patent application no. 202021051801 filed on Nov. 27, 2020, the complete disclosure of which, in their entirety, are hereby incorporated by reference.

### BACKGROUND

### Technical Field

Embodiments herein generally relate to pooling of samples, and more particularly, to a system and method for reducing a number of testings of a population for a high dimensional assay to detect and measure a plurality of analytes.

### Description of the Related Art

High dimensional assay is a method that is used for simultaneous measurement or detection of a plurality of analytes. For example, mass spectrometry test is a type of high dimensional assay that may be used to measure a large number (more than 100) of analytes simultaneously in a single procedure. Mass spectrometry is typically used for newborn screening test and food quality testing. The newborn screening test is useful to prevent mortality and morbidity in young children. It has been made compulsory in developed countries, but is not compulsory in countries of the developing world due to the cost point. Similarly, mass spectrometry test is used to detect adulteration in spices, high levels of pesticide in tea, high levels of antibiotics (in dairy), etc. Such screenings help keep the population at large safe from such contaminants in food. But such screenings are not widely deployed because of cost constraints. At most, samples are randomly tested.

Further, mass spectrometry has throughput limitations. Each sample may take ten minutes or more to analyze. For example, a lab with a single machine has a capacity to test only about 200 samples per day. If 1200 samples need to be tested, this would require the lab to invest in extra machines, which can be a substantial outlay of capital expenditure, making the test expensive.

Pooled testing is used to reduce the cost of screening a large number of population and to increase testing capacity. Pooled testing, also known as “Dorfman pooling”, is an effective method for reducing a number of tests that are required for testing a population when most samples in a population are negative. It works by combining n samples into disjoint groups of k elements (e.g., k=5) each. The samples from groups that are positive are individually retested in a second round. If g groups are positive in a first round, then Dorfman pooling requires a total of n/k+k*g tests, which can be considerably less than n if g is small.

While Dorfman pooling is an effective compression strategy for tests that measure one analyte, it is not effective when a single molecular test measures a large number of analytes. For each analyte, most individual samples will have normal levels of that analyte. However, when samples are pooled, almost certainly at least one sample in the pool has at least one analyte abnormally high. This will increase the value of g to have a value very close to n and a total number of tests (n/k+k*g) is greater than n for every positive integer k. Hence, compression is not achieved by using Dorfman pooling for tests that measure a large number of analytes.

Therefore, there is a need to address the aforementioned technical drawbacks in existing technologies in pooling to reduce a number of testings for a high dimensional assay.

### BRIEF DESCRIPTION OF THE DRAWINGS

The embodiments herein will be better understood from the following detailed descriptions with reference to the drawings, in which:

FIGS. 1A-B illustrate an existing solution for reducing a number of testings using Dorfman pooling.

FIG. 2 illustrates a system for reducing a number of testings for a high-dimensional assay for detecting, identifying, and quantifying a plurality of analytes in a plurality of biological samples according to some embodiments herein;

FIG. 3 is an exemplary schematic illustration of construction of a pooling matrix using the system of FIG. 2, to measure a plurality of analytes using a high dimensional assay according to some embodiments herein:

FIGS. 4A and 4B illustrate a method for reducing a number of testings for a high-dimensional assay for detecting, identifying, and quantifying a plurality of analytes in a plurality of biological samples; and

FIG. 5 is a schematic diagram of computer architecture of a computing device or a molecular computer, in accordance with the embodiments herein.

### SUMMARY OF THE INVENTION

According to a first aspect of the invention, a system for reducing a number of testings for a high-dimensional assay for detecting, identifying, and quantifying a plurality of analytes in a plurality of biological samples is provided. The system includes a memory that stores a set of instructions and a processor that is configured to execute the set of instructions for performing one or more operations. The processor is configured to generate by a sample coding device a pooling matrix for pooling and testing the plurality of biological samples. The pooling matrix indicates a plurality of pools for the plurality of biological samples to be tested and at least two pools for each biological sample. The pooling is performed to include each of the biological samples in the determined at least two pools of the plurality of pools and tests are performed on the plurality of pools. The processor is configured to obtain from a testing machine an output data on completing the high-dimensional assay in each of the plurality of pools with reduced number of testings in the testing machine. For each pool the output data is a row vector that comprises a quantitative vector, a semiquantitative vector or a vector with categorical values indicating an absence, a presence or a category of at least one analyte in the plurality of biological samples. The output data of each pool comprises a measure or the category of each analyte in that pool. The processor is configured to generate a set of linear equations based on the output data and the generated pooling matrix. The processor is configured to converting the set of linear equations into a set of nonlinear equations to solve the set of linear equations using a compressed sensing algorithm. The processor is configured to invoke at least one regularity condition to obtain a unique solution of the set of nonlinear equations to detect, identify, and quantify the plurality of analytes in the plurality of biological samples. The regulatory condition is selected from one of (a) sparsity with respect to a presence or an absence of each analyte separately, or (b) sparsity with respect to a disproportionate number of samples having disproportionately high values for a particular analyte.

According to some embodiments, the processor is configured to detect, identify, and quantify a condition of interest based on the detected, identified, and quantified analytes in the plurality of biological samples. The condition of interest comprises at least one of a condition of quality assurance, a condition of food safety, a medical condition, a medical screening, a drug discovery research, transcriptomics or next generation sequencing (NGS) targeted panels.

According to some embodiments, the testing machine is a polymerase chain reaction (PCR) machine, a high-performance liquid chromatography column (HPLC), microarrays, a next generation sequencing (NGS) device, a mass spectrometer, a nuclear magnetic resonance (NMR) spectroscope, or a Raman spectroscope.

According to some embodiments, the linear equation is y=A x, where,
(i) A=(aᵢⱼ)_{m×n} n is a pooling matrix of dimension m×n. The pooling matrix has a number of rows equal to the number of pools and a number of columns equal to the number of samples. The entry aᵢⱼ of the pooling matrix A in the i^{th} row and j^{th} column determines the amount of sample j that participates in the i^{th} pool.
(ii) x=(xⱼₖ)_{n×d} is a matrix of dimension n×d with entries xⱼₖ, where, j ranges from 1 to n and represents the n samples, and k ranges from 1 to d and represents the d analytes, and xⱼₖ represents the amount of analyte k present in the j^{th} sample. The entries xⱼₖ are unknown and are to be determined by solving the set of linear equations.
(iii) y=(yᵢₖ)_{m×d} is a matrix of dimension m×d with entries yᵢₖ, where, i ranges from 1 to m and k ranges from 1 to d and the matrix y has a number of rows (m) equal to the number of pools and a number of columns (d) equal to the number of analytes being measured in the number of pools. The entries yᵢₖ represent the amount of analyte k present in pool i as determined by the assay or test.

In some embodiments, the processor is configured to convert the linear equation yₖ=A xₖ into a nonlinear equation and then to use the regularity conditions to solve for the matrix x, where xₖ is the k^{th} column of the x matrix and yₖ is the k^{th} column of the y matrix.

In some embodiments, the nonlinear equation is generated based on a plurality of variables that comprise the generated pooling matrix, a plurality of output data of the plurality of pools, and a quantitative measurement of each analyte.

In some embodiments, a statistical correlation between the measurement of the different analytes from previous data is used as a part of the regularity condition.

In some embodiments, the pooling matrix is generated based on an at least one input from a user, wherein the at least input comprises at least one of a name of the assay, and a size of the assay, wherein the size of the assay indicates a total number of biological samples to be tested and a number of biological samples estimated as positive out of the total number of biological samples.

According to a second aspect of the invention, a method for reducing a number of testings for a high-dimensional assay for detecting, identifying, and quantifying a plurality of analytes in a plurality of biological samples is provided. The method includes generating, by a sample coding device, a pooling matrix for pooling and testing the plurality of biological samples. The pooling matrix indicates a plurality of pools for the plurality of biological samples to be tested and at least two pools for each biological sample. A pooling is performed to include each of the biological samples in the determined at least two pools of the plurality of pools and tests are performed on the plurality of pools. The method includes obtaining from a testing machine an output data on completing the high-dimensional assay in each of the plurality of pools with reduced number of testings in the testing machine. For each pool the output data is a row vector that comprises a quantitative vector, a semiquantitative vector or a vector with categorical values indicating an absence, a presence or a category of at least one analyte in the plurality of biological samples. The output data of each pool comprises a measure or the category of each analyte in that pool. The method includes generating a set of linear equations based on the output data and the generated pooling matrix. The method includes converting the set of linear equations into a set of nonlinear equations to solve the set of linear equations using a compressed sensing algorithm. The method includes invoking at least one regularity condition to obtain a unique solution of the set of nonlinear equations to detect, identify, and quantify the plurality of analytes in the plurality of biological samples. The regulatory condition is selected from one of (a) sparsity with respect to a presence or an absence of each analyte separately, or (b) sparsity with respect to a disproportionate number of samples having disproportionately high values for a particular analyte.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

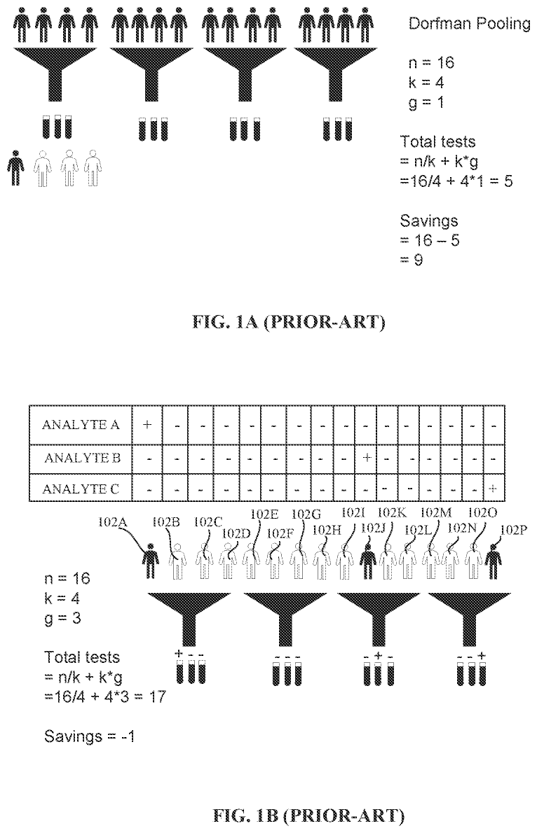

As mentioned, there remains a need for a technique to solve technical drawbacks in existing technologies in pooling. The embodiments herein achieve this by providing a system and method of reducing a number of testings for a high-dimensional assay for detecting, identifying, and quantifying a plurality of analytes in a plurality of biological samples, using a quantitative, non-adaptive and single round pooling. Referring now to the drawings and more particularly to FIGS. 1 through 5, where similar reference characters denote corresponding features consistently throughout the figures, there are shown preferred embodiments.

FIGS. 1A-B illustrate an existing solution for reducing a number of testings using Dorfman pooling. As illustrated in FIGS. 1A-B, n samples are combined into disjoint groups of k elements (e.g., k=5) each. The samples from groups that are positive are individually retested in a second round. If g groups are positive in a first round, then Dorfman pooling requires a total of n/k+k*g tests, which can be considerably less than n if g is small. As illustrated in FIG. 1A, Dorfman pooling is an effective compression strategy for tests that measure one analyte. Dorfman pooling is not effective when a single molecular test measures a large number of analytes, for example an analyte A, an analyte B and an analyte C, as illustrated in FIG. 1B. For each analytes A, B and C, most individual samples have normal levels of that analyte. However, when samples 102A-P are pooled, at least one sample in the pool has at least one analyte abnormally high. This increases the value of g to have a value very close to n and a total number of tests (n/k+k*g)=17 is greater than n=16 for every positive integer k. Hence, as shown in FIG. 1B, savings are negative and compression is not achieved by using Dorfnan pooling for tests that measure a large number of analytes.

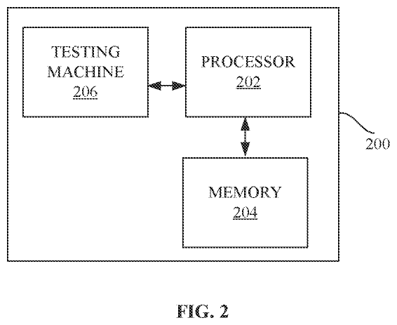

FIG. 2 illustrates a system 200 for reducing a number of testings for a high-dimensional assay for detecting, identifying, and quantifying a plurality of analytes in a plurality of biological samples according to some embodiments herein. The system 200 includes a processor 202 and a memory 204 having stored thereon computer-executable instructions that are executable by the processor 202 to perform one or more operations of the system 200. The system 200 may be at least one of a cloud computing device, a server, or a computing device. The cloud computing device may be a part of a public cloud or a private cloud. The server may be at least one of a standalone server, a server on a cloud, or the like. The computing device may be, but are not limited to, a personal computer, a notebook, a tablet, desktop computer, a laptop, a handheld device, a mobile device, and the like. The system 200 may be at least one of, a microcontroller, a processor, a System on Chip (SoC), an integrated chip (IC), a microprocessor based programmable consumer electronic device, and the like. The system 200 may communicate with an external entity through a network. The network may be, but not limited to, the Internet, a wired network, or a wireless network (a Wi-Fi network, a cellular network, a Wi-Fi Hotspot, Bluetooth, Zigbee and the like).

The system 200 is configured to define a pooling matrix for a plurality of samples to be tested, where each sample is directed to two or more pools. The system 200 may ensure that no two pools include more than one sample in common. Such that, the system 200 reduces the number of testings for the population for measuring the plurality of analytes in a single procedure or single round. The plurality of samples includes the plurality of analytes to be measured.

The pooling matrix includes a plurality of rows and columns. The plurality of columns indicate a number of samples to be tested. The plurality of rows indicate a number of tests or pools to be created for testing of the samples. As an example, A is the pooling matrix which has a dimension of m×n, where m and n are rows and columns of the pooling matrix A. The entry aᵢⱼ of the pooling matrix A in the i^{th} row and j^{th} column determines the amount of sample j that participates in the i^{th} pool. In some embodiments, the pooling matrix may be a sparse matrix or a dense matrix. In an example scenario, samples that have been tested may be numbered as 1, 2, 3 . . . n and indexed by ‘j’, and the pools or tests created for the samples may be numbered as 1, 2, 3 . . . n and indexed by ‘i’. In the example scenario, the system 200 constructs the pooling matrix for testing of the samples as.

$$A = {( {A}_{ij} )}_{m \times n}$$

wherein, Aᵢⱼ=0 indicates that the j^{th} sample is not present in the i^{th} pool, and Aᵢⱼ=1 indicates that the j^{th} sample is present in the i^{th} pool. In some embodiments, the pooling matrix is a part of a preprocessing step in a lab where the samples are combined into pools.

The system 200 is configured to test each pool of the pooling matrix, using the high dimensional assay, for the plurality of analytes. The high dimensional assay may be performed in a testing machine 206 that is associated with the system 200. In some embodiments, the testing machine 206 may be a polymerase chain reaction (PCR) machine, a high-performance liquid chromatography column (HPLC), microarrays, a next generation sequencing (NGS) device, a mass spectrometer, a nuclear magnetic resonance (NMR) spectroscope, or a Raman spectroscope. Each sample that corresponds to each pool of the pooling matrix is transferred into a container for performing the high dimensional assay. In some embodiments, the system 200 tests each pool using one or more high dimensional assays where the one or more high dimensional assays targets different analytes in same sample or same pool. The one or more high dimensional assays may be same technological assays. The one or more high dimensional assays may not be same technological assays. For example, the system 200 runs multiple polymerase chain reaction (PCR) reactions on each pool, each PCR reaction targets different analytes. This might be useful when the same sample needs to be tested for multiple infectious diseases, or for multiple alleles or marker regions on a genome. Hence, the system 200 may use a single highly multiplexed test, or multiple tests that need not even be the same technology. This implies that pooling only needs to be done once, and all this processing may be done downstream in many ways, and all may be solved using subsequent steps that are described below.

The system 200 is configured to determine a row vector (yⱼ) of dimension d for each pool with a result for each analyte in that pool. The row vector (yⱼ) may be a quantitative vector, a semiquantitative vector or a vector with categorical values indicating an absence, a presence or a category of at least one analyte in the plurality of biological samples. The output data of each pool comprises a measure or the category of each analyte in that pool.

The system 200 is configured to determine positive samples from the plurality of samples and thereafter determine the presence of the plurality of analytes in the positive samples, from the row vector (yⱼ). From the row vector (yⱼ), the system 200 uniquely identifies all the positive samples from the row vector (yⱼ), as well as for which analyte the positive samples are positive for.

The system 200 is configured to generate a set of linear equations based on (a) the pooling matrix A that is created for testing of the plurality of samples and (b) the row vector (yⱼ) of dimension d for each analyte in each pool, where yⱼ denotes j^{th} row of y matrix. The linear equation is

$$y = Ax$$

where, x=(xⱼₖ)_{n×d} is a matrix of dimension n×d with entries xⱼₖ, where j ranges from 1 to n and represents the n samples, and k ranges from 1 to d and represents the d analytes, and xⱼₖ represents the amount of analyte k present in the j^{th} sample. The entries xⱼₖ are unknown and are to be determined by solving the set of linear equations, y=(yᵢₖ)_{m×d} is a matrix of dimension m×d with entries yᵢₖ, where i ranges from 1 to m and k ranges from 1 to d and the matrix y has a number of rows (m) equal to the number of pools and a number of columns (d) equal to the number of analytes being measured in the number of pools. The entries yᵢₖ represent the amount of analyte k present in pool i as determined by the assay or test.

The system 200 may convert the linear equation yₖ=A xₖ into a nonlinear equation and then use the regularity conditions to solve for the matrix x.

In one embodiment, the system 200 (i) converts the set of linear equations y=A x into nonlinear equation y=f(A g(x)) by choosing f and g to be log and exp instead of identity functions, where log(a) is understood as (log(a1), log(a2), . . . , log(an)), defining xi log aᵢ that yields b=Ae^{x}, then taking log on both sides, and defining y=log(b) that yields the nonlinear equation

$$y = log ( A {e}^{x} )$$

(ii) solves the nonlinear inverse problem by specifying a regularity condition on x after receiving a noisy measurement y′ of y, a matrix A, to determine quantitative or semi quantitative measurements of the plurality of analytes in each sample. The method of converting the linear equation into the nonlinear equation is useful when the analyte has a very high range of values.

In some embodiments, there is statistical correlation between the measurement of the different analytes which is known from previous data, this may also be used in solving the set of linear equations by using as part of the regularity conditions.

The system 200 is configured to detect, identify, and quantify a condition of interest based on the detected, identified, and quantified analytes in the plurality of biological samples. The condition of interest includes, but is not limited to, a condition of quality assurance, a condition of food safety, a medical condition, a medical screening, a drug discovery research, transcriptomics or next generation sequencing (NGS) targeted panels. The medical condition may include, but is not limited to, an infectious disease, cancer, a genetic disease, an inflammation condition, a metabolic syndrome, a cardiac disease, or diabetes. The medical screening may include, but is not limited to, renal screening, gut microbiome screening, a cardiac screening, pulmonary screening, neurological screening, non-invasive prenatal testing, or a newborn screening. Transcriptomics may include, but is not limited to, bulk transcriptomics or single cell transcriptomics or spatial transcriptomics.

The plurality of analytes may include, but is not limited to, infectious agents, microbial analytes, disease-causing agents, pathogens, contamination agents, blood analytes, chemical species or chemical substances, proteins, nucleic acids, genomic mutations, insertions, and deletions, alleles, marker regions or a biomolecule. The infectious agents may include, but is not limited to, virus, bacteria, fungi, protozoa or helminth. The blood analytes may include, but are not limited to, sodium (Na), potassium (K), urea, glucose, and creatinine. The chemical species or chemical substance is defined as a substance that is composed of chemically identical molecular entities. The proteins are biomolecules comprised of amino acid residues joined together by peptide bonds. The protein may include, but not limited to, antibodies, enzymes, hormones, transport proteins and storage proteins. The nucleic acids include deoxyribonucleic acid (DNA) and ribonucleic acid (RNA). Biomolecules are any molecules that are produced by cells and living organisms.

In one exemplary embodiment, the system 200 is used in newborn screening. The system 200 is used to measure one or more metabolites in blood samples of newborns for determining the presence or absence of disease in the newborns. The system 200 defines a pooling matrix by directing each blood sample to two or more pools while ensuring that no two pools include more than one sample in common. The system 200 further tests each pool in the pooling matrix using a mass spectrometer. The mass spectrometer provides results for each pool as spectra of a signal intensity of detected metabolites as a function of a mass-to-charge ratio. The system 200 constructs the linear equation y=A x based on the pooling matrix and results of each pool from the mass spectrometer. The system 200 determines the matrix x by solving the set of linear equations using compressed sensing algorithm and one or more regularity conditions. The regulatory conditions are selected from one of (a) sparsity with respect to a presence or an absence of each metabolite separately, or (b) sparsity with respect to a disproportionate number of samples having disproportionately high values for a particular metabolite.

In some embodiments, the system 200 further converts the linear equation into a nonlinear equation and determines the quantitative measurements of the one or more metabolites of each sample by solving the nonlinear equation using regulatory condition and nonlinear algorithms to solve for the matrix x. In some embodiments, the one or more metabolites in each blood sample may be identified by correlating known masses (e.g., an entire molecule) to the identified masses or through a characteristic fragmentation pattern.

In another exemplary embodiment, the system 200 is used in food quality testing for determining existence of adulteration in food items like in spices, existence of high levels of pesticide in products like tea, existence high levels of antibiotics in products like dairy products, etc.

In another exemplary embodiment, the system 200 is used to detect or measure presence of one or more pathogens in the plurality of samples.

The embodiments herein are of advantage that an effective sparsity seen when solving for a particular column is the same as the sparsity across the samples for that single analyte. This advantage is available because each sample is sent to multiple pools and is not available in Dorfman pooling method for which the effective sparsity would be determined by the fraction of samples that have positive values for any of the plurality of analytes.

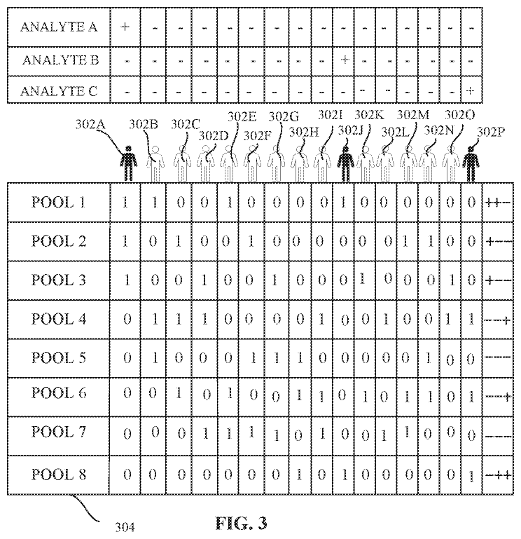

FIG. 3 is an exemplary schematic illustration of construction of a pooling matrix 304 using the system 200 of FIG. 2, to measure the plurality of analytes using a high dimensional assay according to some embodiments herein. The system 200 may receive a request or an instruction to perform pooling and testing of a plurality of the individuals 302A-P to measure the plurality of analytes, from a user. The user may provide the request or instruction via a user device or a user interface of the system 200. The test may be mass spectrometry test. The request may include data including, but not limited to, a unique name for the test (with date and time), a size of the test. The size of the test may indicate a number of samples to be tested, a number of analytes to be tested, and a number of positives expected. As an example, there are d number of analytes to be tested. The plurality of analytes include, but are not limited to, an analyte A, an analyte B, and an analyte C. In the example, d=3.

The system 200 constructs the pooling matrix 304 for pooling and testing based on a given number of samples. The system 200 constructs the pooling matrix 304 by directing each sample to two or more pools and also ensures that no two pools include more than one sample in common. The pooling matrix 304 includes a plurality of rows and columns. The plurality of columns indicate a number of samples to be tested. The plurality of rows indicate a number of tests or pools to be created for testing of the samples. Further to the example, a number of samples or a number of individuals to be tested is 16. The pooling matrix 304 is an 8×16 matrix, which indicates that 16 biological samples may be pooled into 8 pools. The pooling matrix 304 includes 8 rows and 16 columns. The pooling matrix 304 may include entries of 0's and 1's. The entries of 1's in each column indicate that the pools include samples in such columns. The entries of 0's in each column indicate that the pools do not include sample in such columns.

After creating the pooling matrix 304, the user may pipette out or transfer or otherwise allot each sample from each pool into separate reaction wells or containers where a number of reaction wells or containers are equal to the number of pools. For example, the user pipettes out or transfers each sample from a pool 1 of the pooling matrix 304 into a reaction well. Similarly, the user pipettes out or transfers each sample from remaining pools such as pools 2 to 8, into separate reaction wells. The system 200 performs testing of each pool for the plurality of analytes, using the high dimensional assay such as mass spectrometry. The high dimensional assay may be performed in a testing machine 206 that may include a mass spectrometer.

The testing machine 206 may provide signal intensity values (row vector (yⱼ)) of each pool for each analyte. Based on the received signal intensity values of each pool for each analyte, the system 200 determines the pools that are positive and the pools that are negative. The system 200 may convert the signal intensity values that are identified as positive for any one of the analytes into analyte concentration.

The system 200 constructs a linear equation based on the pooling matrix 304 that is created for testing of the plurality of samples 302A-P and the row vector (yⱼ) of dimension d for each analyte in each pool. The linear equation is

$$y = Ax$$

The system 200 determines quantitative measurements of the plurality of analytes in each sample using a compressed sensing algorithm, for example. Lasso or Bayesian inference algorithms like Markov Chain Monte Carlo. The regularity conditions are used to set the prior distributions. In the example, an individual 302A is identified as positive for analyte A, an individual 3021 is identified as positive for analyte B and an individual 302P is identified as positive for analyte C.

In this example, a reduction in the number of tests is achieved, where a number of tests saved is 8 (16−8).

In some embodiments, the system 200 is used for reducing a number of testings for newborn screening using mass spectrometry. The tables below describe data from a validation trial.

| | Sam- | Sam- | Sam- | Sam- | Sam- | Sam- | Sam- | Sam- |
|---|---|---|---|---|---|---|---|---|
| | ple | ple | ple | ple | ple | ple | ple | ple |
| | 1 | 2 | 3 | 4 | 5 | 6 | 7 | 8 |
| Plate | A1 | A2 | A3 | A4 | AS | A6 | A7 | A8 |
| Position | | | | | | | | |
| Plate 1 | 100 | 100 | 100 | 100 | 100 | 100 | 100 | 100 |
| Plate 2 | 200 | 200 | 200 | 200 | 200 | 200 | 200 | 200 |
| Plate 3 | 25 | 25 | 25 | 25 | 25 | 25 | 25 | 25 |
| Plate 4 | 100 | 100 | 100 | 100 | 100 | 100 | 100 | 100 |
| Plate 5 | 100 | 100 | 100 | 100 | 100 | 100 | 100 | 100 |
| Plate 6 | 200 | 200 | 200 | 200 | 200 | 200 | 200 | 200 |
| Plate 7 | 15 | 15 | 15 | 15 | 15 | 15 | 15 | 15 |

The trial was conducted for testing various metabolites (PKU, IRT, 17alpha-OHP, NTSH, GAO, and 40 other metabolites mentioned in the table below (Tandem Mass Spectrometry) with 8 samples whose ground truth values for the metabolites were known.

| SL | | Ref | | SAMPLE_ | SAMPLE_ | SAMPLE_ | SAMPLE_ | SAMPLE_ | SAMPLE_ | SAMPLE_ |
|---|---|---|---|---|---|---|---|---|---|---|
| ORDER | FreeCN | Range | SAMPLE 1 | 2 | 3 | 4 | 5 | 6 | 7 | 8 |
| 1 | FreeCN | >7<125 | 77.25 | 13.93 | 18.57 | 22.12 | 1.95 | 16.05 | 21.75 | 48.52 |
| 2 | C2 | >1.5<80 | 29.05 | 3.79 | 9.41 | 7.39 | 1.29 | 6.62 | 6.5 | 3.36 |
| 3 | C3 | <6.3 | 11.23 | 0.65 | 0.73 | 0.56 | 0.41 | 0.61 | 0.83 | 1.5 |
| 1 | C3DC + | <0.45 | 1.97 | 0.14 | 0.13 | 0.09 | 0.1 | 0.07 | 0.06 | 0.05 |
| | C4-OH | | | | | | | | | |
| 5 | C4 | <1.7 | 3.69 | 0.21 | 0.25 | 0.18 | 0.52 | 0.09 | 0.24 | 0.21 |
| 6 | C4DC | <1.29 | 3.59 | 0.73 | 0.68 | 0.51 | 0.1 | 0.52 | 0.72 | 0.12 |
| 7 | C5 | <1 | 2.91 | 0.18 | 0.09 | 0.08 | 0.05 | 0.07 | 0.07 | 0.1 |
| 8 | C5:1 | <0.9 | 2 | 0.07 | 0.05 | 0.04 | 0.06 | 0.03 | 0.03 | 0.03 |
| 9 | C4OH | <1.29 | 1.78 | 0.11 | 0.1 | 0.08 | 0.08 | 0.06 | 0.04 | 0.05 |
| 10 | C6 | <0.95 | 2.3 | 0.14 | 0.03 | 0.03 | 0.06 | 0.01 | 0.08 | 0.01 |
| 11 | C5-OH | <0.9 | 1.57 | 0.3 | 0.31 | 0.21 | 0.05 | 0.22 | 0.35 | 0.05 |
| 12 | C8:1 | <0.7 | 0.04 | 0.02 | 0.02 | 0.01 | 0.03 | 0.01 | 0.01 | 0.02 |
| 13 | C8 | <0.6 | 1.82 | 0.12 | 0.04 | 0.03 | 0.11 | 0.03 | 0.07 | 0.02 |
| 14 | C10:2 | <0.22 | 0.03 | 0.02 | 0.01 | 0.01 | 0.03 | 0.02 | 0.04 | 0.01 |
| 15 | C10:1 | <0.45 | 0.02 | 0 | 0.01 | 0.01 | 0.11 | 0.02 | 0.01 | 0.03 |
| 16 | C10 | <0.65 | 1.99 | 0.07 | 0.01 | 0.02 | 0.04 | 0.02 | 0.04 | 0.03 |
| 17 | C5DC(C10-OH) | <0.6 | 0.69 | 0.07 | 0.03 | 0.02 | 0.07 | 0.02 | 0.02 | 0.02 |
| 18 | C12:1 | <0.51 | 0.01 | 0.01 | 0.04 | 0.01 | 0.15 | 0.03 | 0.05 | 0.02 |
| 19 | C12 | <0.54 | 2.08 | 0.04 | 0.04 | 0.02 | 0.08 | 0.04 | 0.04 | 0.03 |
| 20 | C14:2 | none | 0.01 | 0 | 0.02 | 0.03 | 0.05 | 0.02 | 0.03 | 0.02 |
| 21 | C14:1 | <0.8 | 1.53 | 0.04 | 0.75 | 0.06 | 3.96 | 0.12 | 3.73 | 0.06 |
| 22 | C14 | <1.2 | 2.51 | 0.07 | 0.22 | 0.14 | 0.39 | 0.1 | 0.26 | 0.19 |
| 23 | C14-OH | <0.2 | 0.02 | 0 | 0 | 0.01 | 0.01 | 0.01 | 0.01 | 0 |
| 24 | C16:1 | <1.4 | 0.04 | 0.03 | 0.02 | 0.03 | 0.03 | 0.04 | 0.01 | 0.07 |
| 25 | C16 | <10 | 12.44 | 1.62 | 0.99 | 2.17 | 0.06 | 2.35 | 0.87 | 2.83 |
| 26 | C16-OH | <0.1 | 1.08 | 0.02 | 0 | 0.04 | 0.07 | 0.01 | 0 | 0 |
| 27 | C18:2 | <0.73 | 0.3 | 0.24 | 0.29 | 0.34 | 0.03 | 0.52 | 0.24 | 0.18 |
| 28 | C18:1 | <7 | 1.02 | 1.37 | 1.07 | 2.12 | 0.04 | 1.94 | 1.1 | 1.1 |
| 29 | C18 | <4 | 4.25 | 0.76 | 0.41 | 0.85 | 0.02 | 0.81 | 0.47 | 0.67 |
| 30 | C18:10H | <0.1 | 0.05 | 0.02 | 0.02 | 0.04 | 0.01 | 0.02 | 0.01 | 0.02 |
| 31 | C18-OH | <0.1 | 1.04 | 0.02 | 0.01 | 0.05 | 0.01 | 0.01 | 0 | 0 |
| 32 | Gly | <745 | 646.05 | 281.47 | 181.08 | 628.81 | 312.58 | 697.18 | 150.04 | 116.77 |
| 33 | Ala | >74<613 | 679.33 | 205.2 | 197.29 | 506.49 | 1214.67 | 377.86 | 287.7 | 159.43 |
| 34 | Val | >41<233 | 279.57 | 69.93 | 52.78 | 187.19 | 353.43 | 128.45 | 85.23 | 65.98 |
| 35 | Leu-Ile | >26<250 | 284.74 | 261.92 | 71.47 | 270.28 | 490 | 220.74 | 124.69 | 87.43 |
| 36 | Met | >1<54 | 168.2 | 2.95 | 11.88 | 32.14 | 8.6 | 9.29 | 4.89 | 2.86 |
| 37 | Cit | >5<60 | 252.92 | 28.28 | 26.89 | 18.09 | 8.49 | 16 | 21.04 | 8.59 |
| 38 | Phe | >21<155 | 290.13 | 43.53 | 30.7 | 140.31 | 331.81 | 90.91 | 41.87 | 33.14 |
| 39 | Tyr | >17<250 | 763.45 | 42.4 | 43.96 | 103.79 | 15.68 | 83.49 | 44.35 | 35.49 |
| 40 | Om | <239 | 362.41 | 65.94 | 54.38 | 142.43 | 77.89 | 111.17 | 72.92 | 56.65 |
| 41 | Arg | <50 | 270.65 | 16.91 | 11.37 | 39.08 | 10.6 | 44.78 | 10.44 | 3.76 |

The 8 samples were pooled 3 times into pools, each pool containing all 8 samples. Example pooling matrix is described in the table below.

| | C1 | C2 | C3 | C4 | C5 | C6 | . . . | C32 |
|---|---|---|---|---|---|---|---|---|
| R1 | R1C1 | R1C2 | R1C3 | R1C4 | R1C5 | R1C6 | | R1C32 |
| R2 | R2C1 | R2C2 | R2C3 | R2C4 | R2C5 | R2C6 | | R2C32 |
| R3 | R3C1 | R3C2 | R3C3 | R3C4 | R3C5 | R3C6 | | R3C32 |
| R4 | R4C1 | R4C2 | R4C3 | R4C4 | R4C5 | R4C6 | | R4C32 |
| R5 | R5C1 | R5C2 | R5C3 | R5C4 | R5C5 | R5C6 | | RSC32 |
| R6 | R6C1 | R6C2 | R6C3 | R6C4 | R6C5 | R6C6 | | R6C32 |
| R24 | R24C1 | R24C2 | R24C3 | R24C4 | R24C5 | R24C6 | | R24C32 |

The matrix above reduces the testing of 768 samples to the testing of only 88 pools which are described below as Row1, Row2, . . . Row24, Col1, Col2, . . . , Col32. Diag1, Diag2, . . . , Diag32.

| Pool | Well | | | | | | | |
|---|---|---|---|---|---|---|---|---|
| Name | Name | Sample Name | | | | | | |
| COL1 | A1 | Sample Name | R1C1 | R2C1 | R3C1 | R4C1 | . . . | R24C1 |
| | | Relative amount | 1 | 1 | 1 | 1 | . . . | 0.0416 |
| COL2 | B1 | Sample Name | R1C2 | R2C2 | R3C2 | R4C2 | . . . | R24C2 |
| | | Relative amount | 1 | 1 | 1 | 1 | . . . | 0.0416 |
| COL3 | C1 | Sample Name | R1C3 | R2C3 | R3C3 | R4C3 | . . . | R24C3 |
| | | Relative amount | 1 | 1 | 1 | 1 | . . . | 0.0416 |
| . . . | | | | | | | | |
| COL32 | H4 | Sample Name | R1C31 | R2C31 | R3C31 | R4C31 | . . . | R24C31 |
| | | Relative amount | 1 | 1 | 1 | 1 | . . . | 1 |
| ROW1 | A5 | Sample Name | R1C1 | R1C2 | R1C3 | R1C4 | . . . | R1C32 |
| | | Relative amount | 1 | 1 | 1 | 1 | . . . | 1 |
| ROW2 | B5 | Sample Name | R2C1 | R2C2 | R2C3 | R2C4 | . . . | R2C32 |
| | | Relative amount | 1 | 1 | 1 | 1 | . . . | 1 |
| ROW3 | C5 | Sample Name | R3C1 | R3C2 | R3C3 | R3C4 | . . . | R3C32 |
| | | Relative amount | 1 | 1 | 1 | 1 | . . . | 1 |
| . . . | | | | | | | | |
| ROW24 | H7 | Sample Name | R24C1 | R24C2 | R24C3 | R24C4 | . . . | R24C32 |
| | | Relative amount | 1 | 1 | 1 | 1 | . . . | 1 |
| DIAG1 | A8 | Sample Name | R1C1 | R2C2 | R3C3 | R4C4 | | R24C24 |
| | | Relative amount | 1 | 1 | 1 | 1 | | 1 |
| DIAG2 | | Sample Name | R1C2 | R2C3 | R3C4 | R4C5 | | R24C25 |
| | | Relative amount | 1 | 1 | 1 | 1 | | 1 |
| DIAG3 | | Sample Name | R1C3 | R2C4 | R3C5 | R4C6 | | R24C26 |
| | | Relative amount | 1 | 1 | 1 | 1 | | 1 |
| . . . | | | | | | | | |
| DIAG32 | | Sample Name | R1C32 | R2C1 | R3C2 | R4C3 | | R24C23 |
| | | Relative amount | 1 | 1 | 1 | 1 | | 1 |

The results show that the numerical value of the pools exhibits linearity with respect to individual sample metabolite values. It also exhibits replicability so that the three pool replicates give rise to nearly the same numerical values. For any fixed analyte, if a metabolite is a heavy-hitter, then all three pool values are above average. This validates the advantages achieved by the system 200 in reducing the number of testings.

In some embodiments, the system 200 is used for reducing a number of testings in next-generation sequencing. As an exemplary scenario, deidentified Fastq files from sequencing 42 samples for a Renal Panel are obtained as ground truth through NGS testing of patient samples. A Fastq file is a text-based file format for storing both a biological sequence (usually nucleotide sequence) and its corresponding quality scores. The fastq files are simulated in silico to simulate an effect of pooling 42 samples according to a pooling matrix into 20 pools. The fastq files from the 20 pools are converted to bam files, and binary alignment map (BAM) files are analyzed at every position with an objective of identifying which variant occurred in which sample. The analysis produced perfect concordance with the ground truth for all variants that are present in less than 7 samples. The dimension of the assay is the length of the BED file for the panel sequencing. A BED file (. bed) is a tab-delimited text file that defines a feature track.

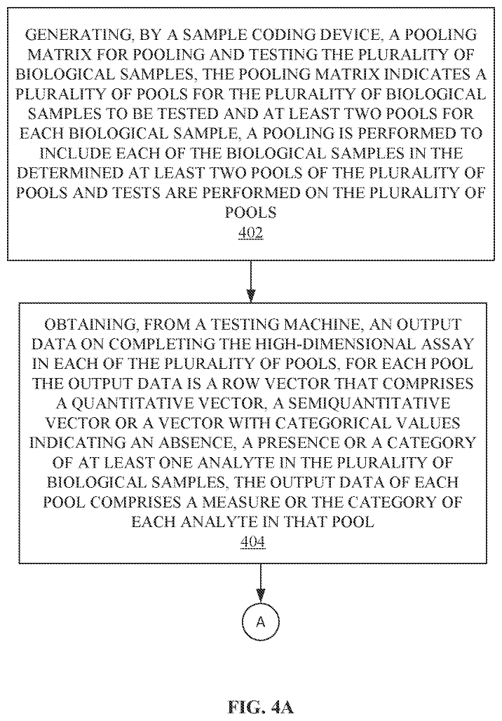

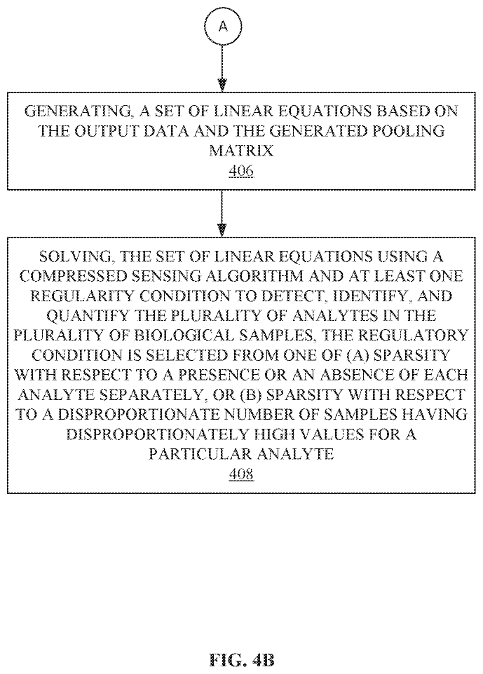

FIGS. 4A and 4B illustrate a method for reducing a number of testings for a high-dimensional assay for detecting, identifying, and quantifying a plurality of analytes in a plurality of biological samples. At step 402, a pooling matrix for pooling and testing the plurality of biological samples is generated using a sample coding device. The pooling matrix indicates a plurality of pools for the plurality of biological samples to be tested and at least two pools for each biological sample. A pooling is performed to include each of the biological samples in the determined at least two pools of the plurality of pools and tests are performed on the plurality of pools. At step 404, an output data on completing the high-dimensional assay in each of the plurality of pools is obtained from a testing machine 206. For each pool the output data is a row vector that comprises a quantitative vector, a semiquantitative vector or a vector with categorical values indicating an absence, a presence or a category of at least one analyte in the plurality of biological samples. The output data of each pool comprises a measure or the category of each analyte in that pool. At step 408, a set of linear equations is generated based on the output data and the generated pooling matrix. At step 410, the set of linear equations are solved using a compressed sensing algorithm and at least one regularity condition to detect, identify, and quantify the plurality of analytes in the plurality of biological samples. The regulatory condition is selected from one of (a) sparsity with respect to a presence or an absence of each analyte separately, or (b) sparsity with respect to a disproportionate number of samples having disproportionately high values for a particular analyte.

The present method is of advantage that the method enables a single testing machine 206 (in case of mass spectrometer) to test a large number of samples, without having to spend extra on additional capital expenditure. Also, there are substantial savings in operational cost as well. This enables an increased supply of such tests, and is able to bring the cost of testing down.

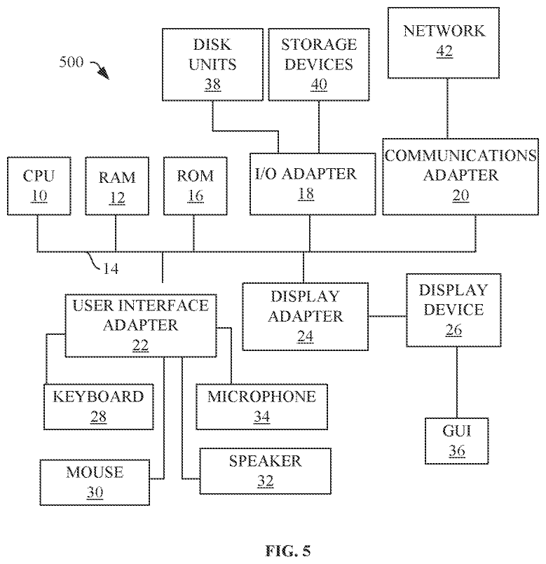

FIG. 5 is a schematic diagram of computer architecture of a computing device or a molecular computer 500, in accordance with the embodiments herein. A representative hardware environment for practicing the embodiments herein is depicted in FIG. 5, with reference to FIGS. 1 through 4B. This schematic drawing illustrates a hardware configuration of a server/computer system/computing device/molecular computer in accordance with the embodiments herein. The system 200 of FIG. 2 may use the computing device or the molecular computer 500 for reducing a number of testings for a population to measure plurality of analytes using a high dimensional assay according to the embodiments herein. The computing device or the molecular computer 500 includes at least one processing device CPU 10 that may be interconnected via system bus 14 to various devices such as a random-access memory (RAM) 12, read-only memory (ROM) 16, and an input/output (I/O) adapter 18. The V/O adapter 18 can connect to peripheral devices, such as disk units 38 and program storage devices 40 that are readable by the system. The system can read the inventive instructions on the program storage devices 40 and follow these instructions to execute the methodology of the embodiments herein. The system further includes a user interface adapter 22 that connects a keyboard 28, mouse 30, speaker 32, microphone 34, and/or other user interface devices such as a touch screen device (not shown) to the bus 14 to gather user input. Additionally, a communication adapter 20 connects the bus 14 to a data processing network 42, and a display adapter 24 connects the bus 14 to a display device 26, which provides a graphical user interface (GUI) 36 of the output data in accordance with the embodiments herein, or which may be embodied as an output device such as a monitor, printer, or transmitter, for example.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents employed herein is for the purpose of description and not of limitation. Therefore, while the embodiments herein have been described in terms of preferred embodiments, those skilled in the art will recognize that the embodiments herein can be practiced with modification within the scope of the appended claims.

1. A system (200) for reducing a number of testings for a high-dimensional assay for detecting, identifying, and quantifying a plurality of analytes in a plurality of biological samples, wherein the system (200) comprises:
a memory (204) that stores a set of instructions;
a processor (202) that is configured to execute the set of instructions for performing one or more operations, characterized in that the processor (202) is configured to:
characterized in that
generate, by a sample coding device, a pooling matrix for pooling and testing the plurality of biological samples, wherein the pooling matrix indicates a plurality of pools for the plurality of biological samples to be tested and at least two pools for each biological sample, wherein a pooling is performed to include each of the biological samples in the determined at least two pools of the plurality of pools and tests are performed on the plurality of pools;
obtain, from a testing machine (206), an output data on completing the high-dimensional assay in each of the plurality of pools with reduced number of testings in the testing machine (206), wherein for each pool the output data is a row vector that comprises a quantitative vector, a semiquantitative vector or a vector with categorical values indicating an absence, a presence or a category of at least one analyte in the plurality of biological samples, wherein the output data of each pool comprises a measure or the category of each analyte in that pool;
generate, a set of linear equations based on the output data and the generated pooling matrix;
convert, the set of linear equations into a set of nonlinear equations to solve the set of linear equations using a compressed sensing algorithm; and
invoke at least one regularity condition to obtain a unique solution of the set of nonlinear equations to detect, identify, and quantify the plurality of analytes in the plurality of biological samples, wherein the regulatory condition is selected from one of (a) sparsity with respect to a presence or an absence of each analyte separately, or (b) sparsity with respect to a disproportionate number of samples having disproportionately high values for a particular analyte.

2. The system (200) as claimed in claim 1, wherein the processor (202) is configured to detect, identify, and quantify a condition of interest based on the detected, identified, and quantified analytes in the plurality of biological samples, wherein the condition of interest comprises at least one of a condition of quality assurance, a condition of food safety, a medical condition, a medical screening, a drug discovery research, transcriptomics or next generation sequencing (NGS) targeted panels.

3. The system (200) as claimed in claim 1, wherein the testing machine (206) is a polymerase chain reaction (PCR) machine, a high-performance liquid chromatography column (HPLC), microarrays, a next generation sequencing (NGS) device, a mass spectrometer, a nuclear magnetic resonance (NMR) spectroscope, or a Raman spectroscope.

4. The system (200) as claimed in claim 1, wherein the linear equation is y=A x, wherein,
(i) A=(aᵢⱼ)_{m×n} is a pooling matrix of dimension m×n, wherein the pooling matrix has a number of rows equal to the number of pools and a number of columns equal to the number of samples, wherein the entry aᵢⱼ of the pooling matrix A in the i^{th} row and j^{th} column determines the amount of sample j that participates in the it pool;
(ii) x=(xⱼₖ)_{n×d} is a matrix of dimension n×d with entries xⱼₖ wherein j ranges from 1 to n and represents the n samples, and k ranges from 1 to d and represents the d analytes, and xⱼₖ represents the amount of analyte k present in the j^{th} sample, wherein the entries xⱼₖ are unknown and are to be determined by solving the set of linear equations; and
(iii) y=(yᵢₖ)_{m×d} is a matrix of dimension m×d with entries yᵢₖ, wherein i ranges from 1 to m and k ranges from 1 to d and the matrix y has a number of rows (m) equal to the number of pools and a number of columns (d) equal to the number of analytes being measured in the number of pools, wherein the entries yₜₖ represent the amount of analyte k present in pool i as determined by the assay or test.

5. The system (200) as claimed in claim 4, wherein the processor (202) is configured to convert the linear equation yₖ=A xₖ into a nonlinear equation and then to use the regularity conditions to solve for the matrix x, wherein xₖ is the k^{th} column of the x matrix and yₖ is the k^{th} column of the v matrix.

6. The system (200) as claimed in claim 5, wherein the nonlinear equation is generated based on a plurality of variables that comprise the generated pooling matrix, a plurality of output data of the plurality of pools, and a quantitative measurement of each analyte.

7. The system (200) as claimed in claim 1, wherein a statistical correlation between the measurement of the different analytes from previous data is used as a part of the regularity condition.

8. The system (200) as claimed in claim 1, wherein the pooling matrix is generated based on an at least one input from a user, wherein the at least input comprises at least one of a name of the assay, and a size of the assay, wherein the size of the assay indicates a total number of biological samples to be tested and a number of biological samples estimated as positive out of the total number of biological samples.

9. A method for reducing a number of testings for a high-dimensional assay for detecting, identifying, and quantifying a plurality of analytes in a plurality of biological samples, wherein the method comprising:
generating, by a sample coding device, a pooling matrix for pooling and testing the plurality of biological samples, wherein the pooling matrix indicates a plurality of pools for the plurality of biological samples to be tested and at least two pools for each biological sample, wherein a pooling is performed to include each of the biological samples in the determined at least two pools of the plurality of pools and tests are performed on the plurality of pools;
obtaining, from a testing machine (206), an output data on completing the high-dimensional assay in each of the plurality of pools with reduced number of testings in the testing machine (206), wherein for each pool the output data is a row vector that comprises a quantitative vector, a semiquantitative vector or a vector with categorical values indicating an absence, a presence or a category of at least one analyte in the plurality of biological samples, wherein the output data of each pool comprises a measure or the category of each analyte in that pool;
generating, a set of linear equations based on the output data and the generated pooling matrix;
converting the set of linear equations into a set of nonlinear equations to solve the set of linear equations using a compressed sensing algorithm; and
invoking at least one regularity condition to obtain a unique solution of the set of nonlinear equations to detect, identify, and quantify the plurality of analytes in the plurality of biological samples, wherein the regulatory condition is selected from one of (a) sparsity with respect to a presence or an absence of each analyte separately, or (b) sparsity with respect to a disproportionate number of samples having disproportionately high values for a particular analyte.
